(19) United States
(12) Patent Application Publication
(10) Pub. No.: US 2026/0290496 A1
(43) Pub. Date: Sep. 24, 2026

(54) PROTEIN SECONDARY STRUCTURE ANALYSIS METHOD BASED ON COMBINATION OF SPECTRUM AND CONVOLUTIONAL NEURAL NETWORK

(71) Applicant: National Center for Nanoscience and Technology, Beijing (CN)

(72) Inventors: Qing DAI, Beijing (CN); Xiaoxia YANG, Beijing (CN); Shiyu YANG, Beijing (CN); Chenchen WU, Beijing (CN)

(73) Assignee: National Center for Nanoscience and Technology, Beijing (CN)

(21) Appl. No.: 19/254,072

(22) Filed: Jun. 30, 2025

(30) Foreign Application Priority Data
Mar. 18, 2025 (CN) 202510322841.2

(51) Int. Cl. G16B 15/20; G06N 3/096; G16B 40/20

(52) U.S. Cl. CPC G16B 15/20; G06N 3/096; G16B 40/20

(57) ABSTRACT

A protein secondary structure analysis method based on combination of a spectrum and a convolutional neural network. The protein secondary structure analysis method includes: designing and calculating protein simulation spectrum data capable of reflecting physical laws; obtaining experimental spectrum data and background spectrum data as a fine-tuning data set; constructing a protein secondary structure percentage prediction model; pre-training the protein secondary structure percentage prediction model; fine-tuning the protein secondary structure percentage prediction model using the fine-tuning data set; obtaining to-be-predicted spectrum data, and predicting a protein secondary structure percentage using the fine-tuned protein secondary structure percentage prediction model.

### CROSS REFERENCE TO THE RELATED APPLICATIONS

This application is based upon and claims priority to Chinese Patent Application No. 202510322841.2, filed on Mar. 18, 2025, the entire contents of which are incorporated herein by reference.

### TECHNICAL FIELD

The present invention relates to the technical field of protein structure analysis, and more specifically to a protein secondary structure analysis method based on combination of a spectrum and a convolutional neural network.

### BACKGROUND

Understanding the secondary structure of proteins at the nanoscale is crucial for elucidating their biological functions and interactions, with implications for fundamental biological processes and advances in nanobiotechnology. For example, protein misfolding and aggregation are often associated with neurodegenerative diseases, and these processes involve transitions in secondary structure, such as from α-helices or Random coils to β-sheets. For example, in the assembly process of silk nanofibril (SNF), the mechanical property of the silk fiber may be adjusted by controlling the percentage of β-sheets to Random coils.

In recent years, deep learning technology has made remarkable progress in the biological field, especially in protein structure prediction. Models such as AlphaFold and RoseTTAFold have successfully predicted the three-dimensional structure of proteins by using protein sequence information and the interactions between amino acids of the proteins. However, due to the limitation of training data, these methods still have limitations in prediction under different environments. In addition, the output three-dimensional structural information usually requires experts with professional biological background to conduct in-depth analysis and quantitative research.

Spectrum technology is an important tool for studying molecular composition and structure. The analysis of the peak shape, peak intensity or peak area of the spectrum may not only reveal molecular structure information, but also be used for quantitative research. Therefore, this analysis has been widely used in protein conformation research. According to the physical principles of the detected signals, spectra used for protein conformation research are often divided into vibrational spectrum and electronic spectrum. Vibrational spectrum (such as Raman spectrum, infrared spectrum, surface-enhanced Raman spectrum, and surface-enhanced infrared spectrum) mainly focuses on the vibration modes of chemical bonds within molecules. The distribution and the change condition of the secondary structure of the protein can be effectively evaluated based on the characteristic spectrum generated by the vibration of the chemical bonds in the molecules. Correspondingly, the electronic spectrum (such as UV-visible spectrum and circular dichroism spectrum) may be used to monitor the folding state, conformational change, and secondary structure percentage of the protein based on the absorption/emission signals generated by the transition process of the electron energy level in the molecule. Different from static analysis, spectroscopy may measure biological samples in situ, thus reflecting the dynamic changes of protein structure in real time. However, due to factors such as overlap of characteristic peaks of protein secondary structure and molecular damping, the characteristic peaks of protein secondary structure often overlap to form a single broad peak, affecting the accurate analysis of the secondary structure, such as the analysis of the amide I band in infrared spectrum. The traditional quantitative analysis method based on the spectrum mainly depends on peak fitting, and the percentage is calculated according to the peak intensity and the area. However, this method is easily affected by the operator's subjective judgment and basis function selection, resulting in unstable fitting results.

To solve these problems, the present invention proposes a new method for predicting protein secondary structure under different environments based on deep learning technology and spectrum technology. The method is based on 1D-CNN. The model receives the measured protein spectrum and directly predicts the secondary structure percentage, thereby achieving accurate quantitative analysis. Given that the performance of the CNN model is highly dependent on the size and diversity of the training dataset, and that it is difficult to obtain a large amount of high-quality experimental spectrum data, a physical information CNN model based on transfer learning is adopted. The core idea is to transfer the learned experience and physical knowledge to an experimental spectrum by using a simulation training data set reflecting the physical laws through transfer learning (i.e., fine-tuning), thereby reducing data requirements and improving training efficiency.

Therefore, it is an issue that needs to be solved urgently by those skilled in the art to provide a protein secondary structure analysis method based on combination of a spectrum and a convolutional neural network to solve the difficulties existing in the prior art.

### SUMMARY

In view of this, the present invention provides a protein secondary structure analysis method based on combination of a spectrum and a convolutional neural network, which is used to solve the technical problems in the prior art.

To achieve the foregoing objectives, the following technical solutions are provided in the present invention.

A protein secondary structure analysis method based on combination of a spectrum and a convolutional neural network includes the following steps:
S1. designing and calculating protein simulation spectrum data as a pre-training data set capable of reflecting physical laws;
S2. obtaining experimental spectrum data and background spectrum data as a fine-tuning data set;
S3. constructing a protein secondary structure percentage prediction model;
S4. pre-training the protein secondary structure percentage prediction model;
S5. fine-tuning the protein secondary structure percentage prediction model using the fine-tuning data set in the S2; and
S6. obtaining to-be-predicted spectrum data, and predicting a protein secondary structure percentage using the fine-tuned protein secondary structure percentage prediction model.

According to the method, optionally, the designing protein simulation spectrum data in the S1 includes the following physical conditions:
protein thickness;
center peak position of characteristic peak; and
full width at half maximum (FWHM) of characteristic peak.

According to the method, optionally, the specific content of calculating protein simulation spectrum data in the S1 is as follows:

the calculated protein simulation spectrum data is used to pre-train a model, called a pre-training data set, and the pre-training data set is expanded by adding random noise into the protein simulation spectrum data.

According to the method, optionally, the adding random noise into the protein simulation spectrum data specifically includes:
for each data point Si in the spectrum S, generating a corresponding Gaussian noise ni, wherein the Gaussian noise ni is sampled from a normal distribution with a mean μ=0 and a specified standard deviation σ, the standard deviation controls an intensity of the noise, and then obtaining a noise-adjusted data point Si′ adding the generated Gaussian noise ni to each data point Si in the spectrum S, wherein the formula is as follows:

$$s = [ {s}_{1} , {s}_{2} , … , {s}_{N} ]$$

$${n}_{i} ∼ N ( μ , {σ}^{2} )$$

$${s}_{i}^{′} = {s}_{i} + {n}_{i} .$$

According to the method, optionally, the specific content of constructing a protein secondary structure percentage prediction model in the S3 is as follows:
the protein secondary structure percentage prediction model is constructed by adopting a Pytorch deep learning framework, a core network framework is a convolutional neural network CNN, and a quantity of filters is set in a mode of increasing firstly and then reducing in a convolutional layer;
the convolutional layer uses an activation function and combines batch normalization to accelerate training convergence, the protein secondary structure percentage prediction model maps the extracted features by at least one fully connected layer after completing feature extraction of the convolutional layer, and a final output layer consists of 3 neurons, corresponding to three secondary structure percentages of proteins.

According to the method, optionally, a parameter and a structure of the protein secondary structure percentage prediction model are dynamically adjusted when the convolutional neural network CNN deals with different training sets.

According to the method, optionally, the pre-training the protein secondary structure percentage prediction model in the S4 specifically includes: firstly, dividing the obtained pre-training data set into a training set, a verification set and a test set in a ratio of 6:2:2, normalizing the training set by a sklearn library, processing the verification set and the test set by the same normalization parameters, and finally storing pre-training weights obtained in a pre-training stage for subsequent fine-tuning.

According to the method, optionally, the specific content of fine-tuning the protein secondary structure percentage prediction model using the fine-tuning data set in the S2 in the S5 is as follows:
the fine-tuning data set is divided into a training set, a verification set and a testing set in a ratio of 6:2:2, normalization processing is performed by using sklearn, the obtained pre-training weight is used as an initial weight in the training process, iterative optimization is performed on the basis, finally, the protein secondary structure percentage prediction model is evaluated by using the testing set, the generalization performance of the fine-tuned protein secondary structure percentage prediction model is measured, and the fine-tuned new weight is used for a final prediction task.

It may be known from the foregoing technical solutions that, compared with the prior art, the present invention provides a protein secondary structure analysis method based on combination of a spectrum and a convolutional neural network, which has the following beneficial effects:
(1) The efficient analysis of protein secondary structure is achieved:
The present invention breaks through the limitation of traditional spectrum technology that relies on peak fitting for secondary structure analysis, and combines 1D-CNN and spectrum technology to achieve accurate and efficient analysis of protein secondary structure under different environments.
(2) Professional background requirements are lowered, and the operation experience is convenient:
Since the CNN model is used to predict the protein secondary structure, the operator can obtain the percentage of the secondary structure without having a deep biological professional background; this characteristic enables researchers in the fields of bioengineering, drug and vaccine design to use the technology more conveniently, and lowers the threshold of professional knowledge.
(3) Broad applicability of spectroscopy:
The present invention has wide applicability. In theory, any spectroscopy technology that can reflect protein structural information may be applied to the method, not limited to vibrational spectroscopy and electronic spectroscopy; for example, far-infrared spectroscopy, mass spectroscopy, and the like can also provide structural information. The present method may achieve technical transfer, further enhancing the versatility in a variety of experimental and research environments.
(4) The reliance on large amounts of experimental data is reduced, and the efficiency of model training is improved:
By designing a simulation training data set reflecting physical laws and combining a transfer learning technology, the present invention greatly reduces the requirement on a large amount of experimental spectrum data, which not only reduces the experiment cost, but also obviously improves the training efficiency and effect of the CNN model, so that the CNN model can be quickly adapted to complex noise and variation factors in the experiment.

### BRIEF DESCRIPTION OF THE DRAWINGS

To more clearly illustrate the technical solutions in the embodiments of the present invention or in the prior art, the drawings required to be used in the description of the embodiments or the prior art are briefly introduced below. It is obvious that the drawings in the description below are merely embodiments of the present invention, and those of ordinary skill in the art can obtain other drawings according to the drawings provided without creative efforts.

FIG. 1 is a flow chart of a protein secondary structure analysis method based on combination of a spectrum and a convolutional neural network according to the present invention;

FIG. 2 is an infrared spectrum diagram of protein simulation and experimental measurement according to the present invention;

FIG. 3 is a diagram of training loss and verification loss in a pre-training stage of a CNN model according to the present invention;

FIG. 4 is a diagram of training loss and verification loss in a fine-tuning stage of a CNN model according to the present invention;

FIG. 5 is a comparison diagram of the prediction accuracy of a pre-trained weight and a fine-tuned weight provided by the present invention;

FIG. 6 is a comparison diagram of an SNF secondary structure percentage predicted by CNN according to the present invention and a peak fitting result;

FIG. 7 is an infrared spectrum diagram of SNF at different graphene Fermi levels measured experimentally according to the present invention; and

FIG. 8 is a diagram of a secondary structure percentage predicted by a CNN model based on spectrum data in FIG. 7 according to the present invention.

### DETAILED DESCRIPTION OF THE EMBODIMENTS

The following clearly and completely describes the technical solutions in embodiments of the present invention with reference to the accompanying drawings in embodiments of the present invention. It is clear that the described embodiments are merely a part rather than all of embodiments of the present invention. Based on the embodiments of the present invention, all other embodiments obtained by those of ordinary skill in the art without creative efforts shall fall within the protection scope of the present invention.

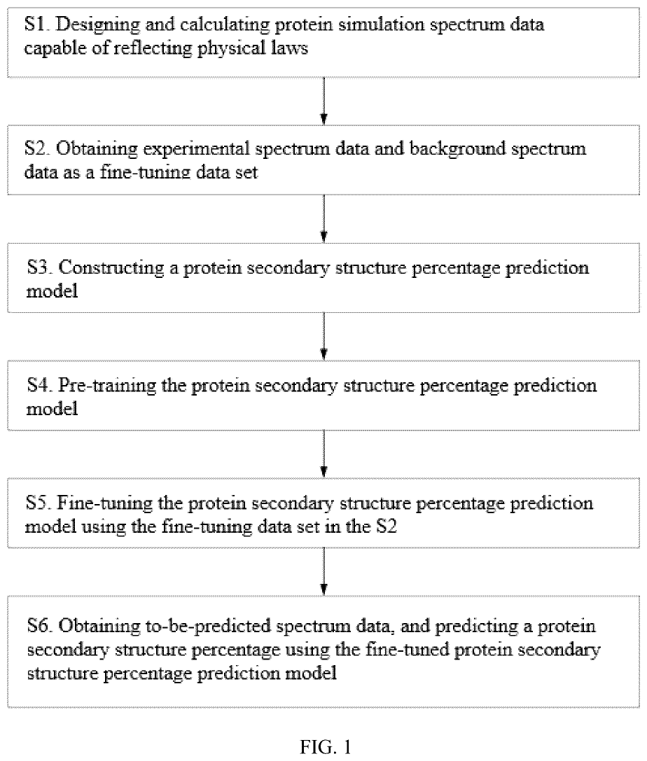

Referring to FIG. 1, the present invention discloses a protein secondary structure analysis method based on combination of a spectrum and a convolutional neural network, which includes the following steps:
S1. Designing and calculating protein simulation spectrum data capable of reflecting physical laws.
S2. Obtaining experimental spectrum data and background spectrum data as a fine-tuning data set.

Specifically, in an experimental environment, experimental spectrum data of protein is collected, background spectrum data (an environmental spectrum without protein, such as an aqueous solution spectrum in a liquid stage) in a corresponding environment is obtained, the two spectra are jointly used as a fine-tuning data set, and a pre-training data set is expanded by adding random noise.
S3. Constructing a protein secondary structure percentage prediction model.
S4. Pre-training the protein secondary structure percentage prediction model.
S5. Fine-tuning the protein secondary structure percentage prediction model using the fine-tuning data set in the S2.
S6. Obtaining to-be-predicted spectrum data, and predicting a protein secondary structure percentage using the fine-tuned protein secondary structure percentage prediction model.

Further, the designing protein simulation spectrum data in the S1 includes, but is not limited to, the following physical conditions:
protein thickness: 10 thicknesses are designed for each protein;
graphene Fermi level: 10 graphene Fermi levels are used for each protein;
center peak positions of characteristic peaks: β-sheets, Random coils, Turns; and
full width at half maximum (FWHM) of characteristic peak.

Specifically, the simulated infrared spectra of 31 proteins on graphene nanoribbons are calculated using COMSOL Multiphysics 6.0. To accelerate model learning and reduce the need for training data, the dataset is designed with multiple physical conditions so as to better reflect physical laws. Specifically, an amide I band in an infrared spectrum of proteins contains characteristic peaks of three secondary structures, namely β-sheets, Random coils and Turns. In this embodiment, the graphene nanoribbons achieve surface enhanced infrared spectroscopy effect by exciting plasmon modes. Graphene plasmons are localized electromagnetic wave modes formed by collective electron oscillations on the graphene surface, which are usually strongly localized and tunable in the mid-infrared to terahertz bands. Compared with traditional metal plasmons, graphene plasmons have lower losses, and the resonant frequency of the graphene plasmons may be dynamically adjusted by adjusting the Fermi level of graphene, making the graphene plasmons suitable for precise control in different spectral ranges. In this embodiment, the graphene plasmon mode is coupled with the vibration mode of the protein amide I band to form Fano resonance, which makes the characteristic peak of the secondary structure clearer and improves the sensitivity of the spectral signal.

Further, the specific content of calculating protein simulation spectrum data in the S1 is as follows:
the calculated protein simulation spectrum data is used to pre-train a model, called a pre-training data set, and the pre-training data set is expanded by adding random noise into the protein simulation spectrum data.

Specifically, in order to expand the pre-training data set and enhance the generalization performance of the protein secondary structure percentage prediction model, the protein secondary structure percentage prediction model can learn more robust and invariant features, and meanwhile, irrelevant tiny changes are ignored, and the pre-training data set is expanded by adding random noise into the protein simulation spectrum data.

Further, the adding random noise into the protein simulation spectrum data specifically includes:
for each data point Si in the spectrum S, generating a corresponding Gaussian noise ni, wherein the Gaussian noise ni is sampled from a normal distribution with a mean μ=0 and a specified standard deviation σ, the standard deviation controls an intensity of the noise, and then obtaining a noise-adjusted data point Si′ adding the generated Gaussian noise ni to each data point Si in the spectrum S, wherein the formula is as follows:

$$s = [ {s}_{1} , {s}_{2} , … , {s}_{N} ]$$

$${n}_{i} ∼ N ( μ , {σ}^{2} )$$

$${s}_{i}^{′} = {s}_{i} + {n}_{i} .$$

Further, the specific content of constructing a protein secondary structure percentage prediction model in the S3 is as follows:
the protein secondary structure percentage prediction model is constructed by adopting a Pytorch deep learning framework, a core network framework is a convolutional neural network CNN, and a quantity of filters is set in a mode of increasing firstly and then reducing in a convolutional layer;
the convolutional layer uses an activation function and combines batch normalization to accelerate training convergence, the protein secondary structure percentage prediction model maps the extracted features by at least one fully connected layer after completing feature extraction of the convolutional layer, and a final output layer consists of 3 neurons, corresponding to three secondary structure percentages of proteins.

Specifically, the convolutional neural network CNN contains four convolutional layers, the number of filters (convolution kernels) first increases and then decreases, and the size of the convolution kernel remains consistent.

Specifically, to retain the spectral information to the greatest extent, the protein secondary structure percentage prediction model does not use a pooling layer, but instead uses a strided convolution with a stride of 2 to reduce the data dimension, thereby reducing information loss while maintaining the integrity of the spectral features. Each convolutional layer uses an activation function to enhance the nonlinear expression capability of the model. Similarly, to ensure that the protein secondary structure percentage prediction model outputs non-negative values, the ReLU activation function is applied in both the fully connected layer and the output layer. During the training process, the learning rate decays from 0.01. In addition, the loss function is optimized by adding constraints on the basis of the mean square error (MSE) loss function to ensure that the sum of the output ratios is close to 100, or equal to 0 in the case of background spectrum, so that the physical rationality of a prediction result is improved. The optimizer uses Adam to enhance the stability and convergence speed of training.

Further, a parameter and a structure of the protein secondary structure percentage prediction model are dynamically adjusted when the convolutional neural network CNN deals with different training sets.

Further, the pre-training the protein secondary structure percentage prediction model in the S4 specifically includes: firstly, dividing the obtained pre-training data set into a training set, a verification set and a test set in a ratio of 6:2:2, normalizing the training set by a sklearn library, processing the verification set and the test set by the same normalization parameters, and finally storing pre-training weights obtained in a pre-training stage for subsequent fine-tuning.

Specifically, the sklearn library is used to normalize the training set, and the same normalization parameters are used to process the validation set and the test set to ensure the consistency of data distribution. During the pre-training process, feature extraction and parameter optimization are achieved by learning the physical laws contained in the protein simulation spectrum data of the training set and the relationship between the physical laws and the secondary structure of the protein. Meanwhile, during the iterative training process, the weight parameters are continuously optimized based on the performance of the validation set to improve the generalization capability of the verification set. Finally, the prediction performance of the protein secondary structure percentage prediction model is evaluated using the test set to measure the final effect of pre-training. Finally, the weight parameters (pre-training weights) obtained in the pre-training stage are saved for subsequent fine-tuning.

Further, the specific content of fine-tuning the protein secondary structure percentage prediction model using the fine-tuning data set in the S2 in the S5 is as follows:
the fine-tuning data set is divided into a training set, a verification set and a testing set in a ratio of 6:2:2, normalization processing is performed by using sklearn, the obtained pre-training weight is used as an initial weight in the training process, iterative optimization is performed on the basis, finally, the protein secondary structure percentage prediction model is evaluated by using the testing set, the generalization performance of the fine-tuned protein secondary structure percentage prediction model is measured, and the fine-tuned new weight is used for a final prediction task.

Specifically, the obtained experimental spectrum data and background spectrum data are used as a fine-tuning data set to adjust the protein secondary structure percentage prediction model so as to adapt to errors, environmental changes or other experimental factors possibly existing in actual measurement, and therefore the adaptability and the generalization capability of the protein secondary structure percentage prediction model are improved. Furthermore, the fine-tuning stage uses the same model structure as pre-training stage. During the training process, the obtained pre-trained weights are used as the initial weights, and iterative optimization is performed on this basis. To further improve the feature extraction capability of the protein secondary structure percentage prediction model, the high-order convolutional layers (the third and fourth layers) and the fully connected layers are unfrozen, so that the weights can be updated in the fine-tuning process, and the feature distribution of experimental spectrum data can be fully learned. The loss function and optimizer settings in the fine-tuning stage are consistent with those in the pre-training stage. Finally, the protein secondary structure percentage prediction model is evaluated by using the test set to measure the generalization performance of the fine-tuned protein secondary structure percentage prediction model. The new weights after fine-tuning (fine-tuned weights) will be used for the final prediction task.

In a specific embodiment, to verify the application capability of 1D-CNN in predicting protein secondary structure, the secondary structure of silk nanofibril (SNF) in aqueous solution characterized by surface enhanced infrared spectroscopy based on graphene plasmons is taken as an example:

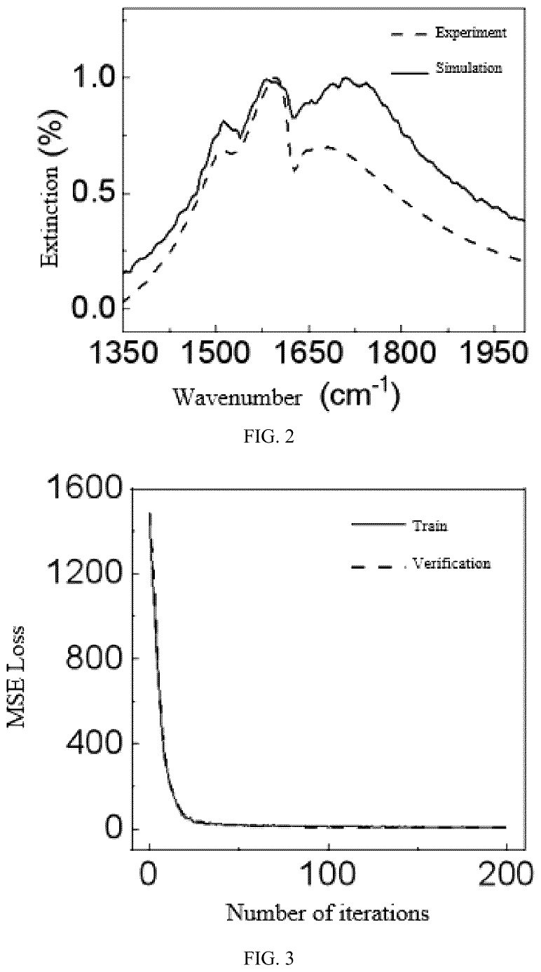

The experimental spectra are measured using Fourier transform infrared spectroscopy (FTIR) and using a Bruker Hyperion 2000 infrared microscope. The simulated spectrum is generated by the finite element method (COMSOL Multiphysics 6.0), as shown in FIG. 2.

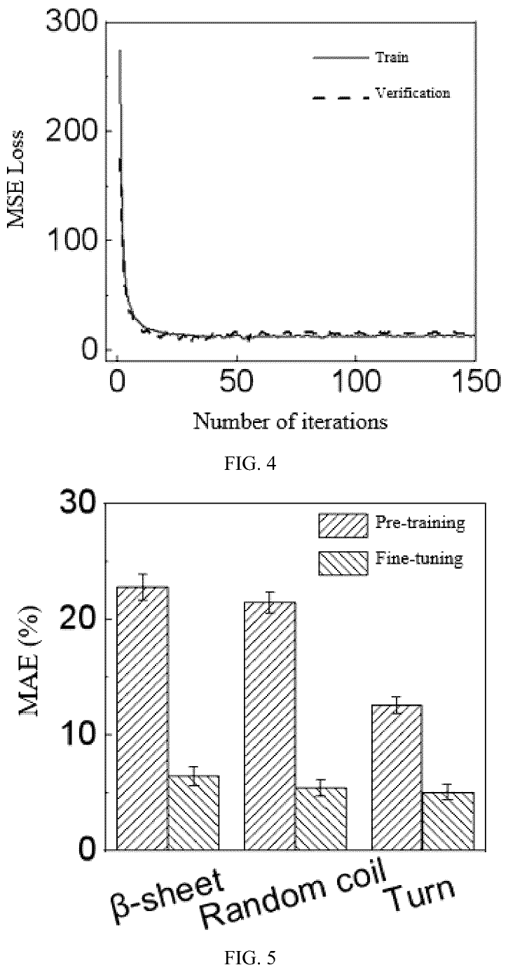

In terms of model training, the mean square error (MSE) loss of the CNN training set and the validation set is first recorded in the pre-training stage, as shown in FIG. 3. After 150 training cycles, the model begins to converge, the training loss stabilized at around 4, and the validation loss stabilized at around 7. The gap between the two is small, indicating that the model is not overfitted. In the fine-tuning stage, as shown in FIG. 4, after 50 iterations, the training and validation losses converge to 13 and 15 respectively, and the gap between the two is still small, further verifying that overfitting does not occur during the fine-tuning process.

To evaluate the effectiveness of the transfer learning strategy, the prediction accuracy of using only pre-trained weights and fine-tuned weights is compared. As shown in FIG. 5, if only pre-trained weights are used, the mean absolute error (MAE) of β-sheet is 22.8%±1.1%, Random coil is 21.4%±0.9%, and Turn is 12.6%±0.7%. After fine-tuning, the MAE of β-sheet drops to 6.4%±0.8%, Random coil drops to 5.5%±0.7%, and Turn drops to 5.1%±0.7%, indicating that transfer learning plays a significant role in improving model efficiency and prediction accuracy.

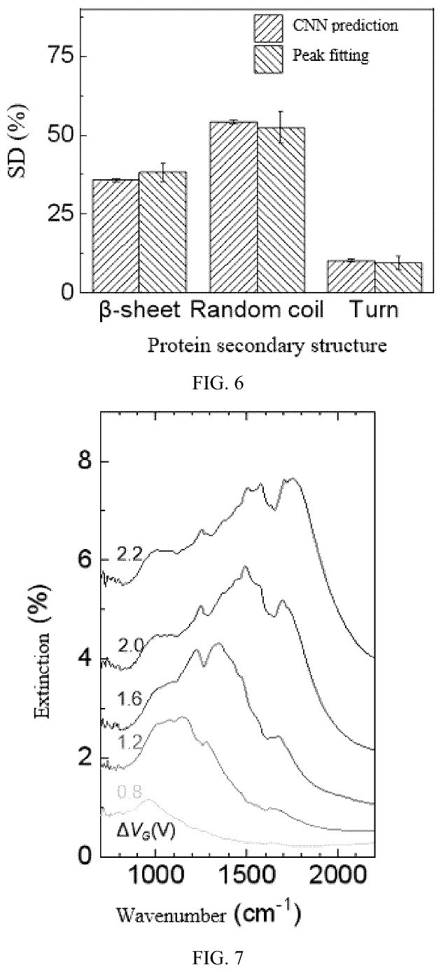

To further verify the reliability of the model, the present invention compares the performance of traditional peak fitting and CNN prediction in the analysis of the secondary structure percentage (SD) of SNF. The results show that the SDs of CNN predictions are: β-sheet=35.6%±0.4%, Random coil=54.2%±0.6%, and Turn=10.2%±0.5%. In contrast, the results obtained by peak fitting analysis of the SNF spectrum are: β-sheet=38.1%±3.0%, Random coil=52.4%±5.1%, and Turn=9.5%±2.2%. The data obtained by the two methods are highly consistent, indicating the accuracy and reliability of the CNN prediction method in secondary structure analysis, as shown in FIG. 6.

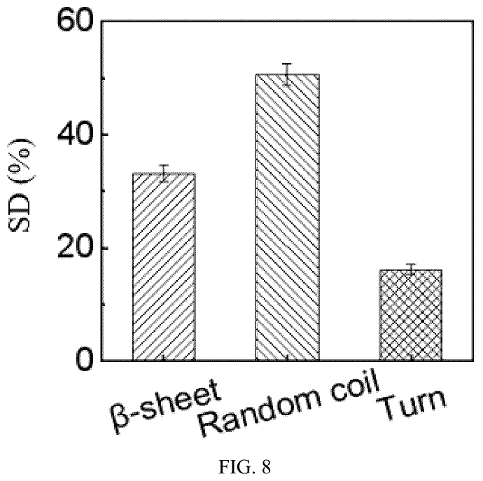

FIG. 7 shows the infrared spectra of SNF in aqueous solution at different graphene Fermi levels (ΔVG) measured in the experiment. The trained CNN prediction model is used to analyze the SNF spectrum in FIG. 7 to obtain the SD prediction results. The model predicts that the 0β-sheet percentage is approximately 33.1%±1.5%, the Random coil percentage is approximately 50.7%±2.0%, and the Turn percentage is approximately 16.2%±0.9%. As shown in FIG. 8, the prediction results show that the model can effectively analyze the secondary structure of proteins, further verifying the practical value of this model in protein structure analysis.

The present invention has broad potential for application beyond the specific protein types analyzed in the present invention. By tailoring the way the spectrum measures different types of proteins, the method can be extended to analyze small secondary structural changes in various protein families and complex protein-biomolecule systems. For example, this method may be applied to amyloid peptide aggregation in neurodegenerative diseases, structure-function relationships of insulin receptors in diabetes, conformational changes in membrane transporters, protein-drug interactions in cancer therapy, and enzyme-substrate interactions in metabolic processes. CNN based on transfer learning significantly reduces the demand for experimental spectra and improves training efficiency. This breakthrough lays an important foundation for the application of AI-driven protein dynamics and structure prediction models in complex biological environments, and opens up new paths for innovation in drug discovery, disease diagnosis and bionic manufacturing.

The embodiments in the specification are all described in a progressive manner, and each embodiment focuses on differences from other embodiments, and portions that are the same and similar between the embodiments may be referred to each other.

The above description of the disclosed embodiments enables those skilled in the art to implement or use the present application. Various modifications to these embodiments will be readily apparent to those skilled in the art, and the generic principles defined herein may be applied to other embodiments without departing from the spirit or scope of the present application. Thus, the present application is not intended to be limited to these embodiments shown herein but is to be accorded the broadest scope consistent with the principles and novel features disclosed herein.

What is claimed is:

1. A protein secondary structure analysis method based on a combination of a spectrum and a convolutional neural network, comprising the following steps:
S1, designing and calculating protein simulation spectrum data as a pre-training data set configured for reflecting physical laws;
S2, obtaining experimental spectrum data and background spectrum data as a fine-tuning data set;
S3, constructing a protein secondary structure percentage prediction model;
S4, pre-training the protein secondary structure percentage prediction model to obtain a pre-trained protein secondary structure percentage prediction model;
S5, fine-tuning the pre-trained protein secondary structure percentage prediction model to obtain a fine-tuned protein secondary structure percentage prediction model using the fine-tuning data set in the step S2; and
S6, obtaining to-be-predicted spectrum data, and predicting a protein secondary structure percentage using the fine-tuned protein secondary structure percentage prediction model.

2. The protein secondary structure analysis method based on the combination of the spectrum and the convolutional neural network according to claim 1, wherein the designing the protein simulation spectrum data in the step S1 comprises the following physical conditions:
a protein thickness;
a center peak position of a characteristic peak; and
a full width at half maximum (FWHM) of the characteristic peak.

3. The protein secondary structure analysis method based on the combination of the spectrum and the convolutional neural network according to claim 1, wherein a content of calculating the protein simulation spectrum data in the step S1 is as follows:
the protein simulation spectrum data is configured to pre-train the protein secondary structure percentage prediction model, called the pre-training data set, and the pre-training data set is expanded by adding a random noise into the protein simulation spectrum data.

4. The protein secondary structure analysis method based on the combination of the spectrum and the convolutional neural network according to claim 3, wherein the adding the random noise into the protein simulation spectrum data comprises:
for each data point Si in a spectrum S, generating a corresponding Gaussian noise ni, wherein the corresponding Gaussian noise ni is sampled from a normal distribution with a mean μ=0 and a predetermined standard deviation σ, the predetermined standard deviation controls an intensity of the random noise, and then obtaining a noise-adjusted data point Si′ adding the corresponding Gaussian noise ni to each data point Si in the spectrum S, wherein a formula is as follows:
$$s = [ {s}_{1} , {s}_{2} , … , {s}_{N} ]$$
$${n}_{i} ∼ N ( μ , {σ}^{2} )$$
$${s}_{i}^{′} = {s}_{i} + {n}_{i} .$$

5. The protein secondary structure analysis method based on the combination of the spectrum and the convolutional neural network according to claim 1, wherein a content of constructing the protein secondary structure percentage prediction model in the step S3 is as follows:
the protein secondary structure percentage prediction model is constructed by adopting a Pytorch deep learning framework, a core network framework is the convolutional neural network CNN, and a quantity of filters is set in a mode of increasing firstly and then reducing in a convolutional layer;
the convolutional layer uses an activation function and combines batch normalization to accelerate training convergence, the protein secondary structure percentage prediction model maps extracted features by at least one fully connected layer after completing feature extraction of the convolutional layer, and a final output layer comprises 3 neurons, corresponding to three secondary structure percentages of proteins.

6. The protein secondary structure analysis method based on the combination of the spectrum and the convolutional neural network according to claim 5, wherein a parameter of the protein secondary structure percentage prediction model and a structure of the protein secondary structure percentage prediction model are dynamically adjusted when the convolutional neural network CNN deals with different training sets.

7. The protein secondary structure analysis method based on the combination of the spectrum and the convolutional neural network according to claim 3, wherein the pre-training the protein secondary structure percentage prediction model in the step S4 comprises: firstly, dividing the pre-training data set into a first training set, a first verification set and a first test set in a ratio of 6:2:2, normalizing the first training set by a sklearn library, processing the first verification set and the first test set by same normalization parameters, and finally storing a pre-training weight obtained in a pre-training stage for subsequent fine-tuning.

8. The protein secondary structure analysis method based on the combination of the spectrum and the convolutional neural network according to claim 7, wherein a content of fine-tuning the pre-trained protein secondary structure percentage prediction model using the fine-tuning data set in the step S2 in the step S5 is as follows:
the fine-tuning data set is divided into a second training set, a second verification set and a second testing set in the ratio of 6:2:2, normalization processing is performed by using the sklearn library, the pre-training weight is used as an initial weight in a training process, iterative optimization is performed on a basis of the initial weight, finally, the fine-tuned protein secondary structure percentage prediction model is evaluated by using the second testing set, a generalization performance of the fine-tuned protein secondary structure percentage prediction model is measured, and a fine-tuned new weight is configured for a final prediction task.
